(19) United States
(12) Patent Application Publication
(10) Pub. No.: US 2026/0157397 A1
(43) Pub. Date: Jun. 11, 2026

(54) YEAST FERMENTATION BROTH OF SALIDROSIDE FOR ANIMAL HUSBANDRY APPLICATIONS

(71) Applicant: RIANLON CORPORATION, Tianjin (CN)

(72) Inventors: Lu Liu, Tianjin (CN); Xiabin Shi, Tianjin (CN); Dalong Zhang, Tianjin (CN); Shu Wang, Tianjin (CN); Jinping Tian, Tianjin (CN); Chunguang Sun, Tianjin (CN); Haiping Li, Tianjin (CN)

(21) Appl. No.: 19/389,507

(22) Filed: Nov. 14, 2025

Related U.S. Application Data
(60) Provisional application No. 63/730,188, filed on Dec. 10, 2024.

(51) Int. Cl. A23K 10/16; A23K 20/121; C12N 1/18; C12R 1/865

(52) U.S. Cl. CPC A23K 10/16; A23K 20/121; C12N 1/185; C12R 2001/865

(57) ABSTRACT

The present disclosure relates to yeast fermentation broths of salidroside for animal husbandry applications, animal feed formulations incorporating the same, and methods of forming and administering the same.

### CROSS-REFERENCE TO RELATED APPLICATION(S)

This application claims the benefit of priority of U.S. Provisional Patent Application No. 63/730,188, filed Dec. 10, 2024, the disclosure of which is hereby incorporated by reference herein in its entirety.

### TECHNICAL FIELD

The present disclosure is directed to animal husbandry applications, and in particular, to additives for use in animal feed formulations.

### BACKGROUND

During the growth and development of animals, nutrients including protein, amino acids, minerals, and trace elements are required. When ordinary feed cannot meet the growth needs of animals, the addition of feed additives containing trace elements or other nutrients can promote rapid animal growth. Further, such additives can also increase the palatability of the feed, promote animal development, treat animal diseases, and reduce production costs.

Non-nutritional feed additives include microecological preparations, enzyme preparations, traditional Chinese medicine preparations, and their extracts. Plant extracts have the ability to improve animal immune function and environmental stress response. However, from a process perspective, the components of plant extracts are relatively complex and difficult to produce as costs of extraction are high. Some components are difficult to obtain pure products, making it infeasible to carry out large-scale industrial production. From the perspective of product standards, the active ingredient content and extraction quality of medicinal materials from different varieties and origins vary greatly.

### OBJECTS AND SUMMARY OF THE INVENTION

Certain embodiments of the present invention are directed to a feed additive comprising a fermentation broth of a microbial strain.

In certain embodiments, the microbial strain is a yeast strain.

In certain embodiments, the yeast strain is Saccharomyces cerevisiae or a modified version thereof.

In certain embodiments, the feed additive maintains at least 95% chemical stability after about six months storage at room temperature or approximately 20-25° C.

In certain embodiments, the fermentation broth comprises salidroside.

Certain embodiments of the present invention are directed to an animal feed formulation comprising a feed additive as disclosed herein, and at least one carrier excipient.

Certain embodiments of the present invention are directed to an animal feed formulation comprising salidroside present, e.g., from about 1 mg/kg to about 150 mg/kg, from about 1 mg/kg to about 100 mg/kg, from about 10 mg/kg to about 100 mg/kg, or from about 10 mg/kg to about 20 mg/kg, from about 20 mg/kg to about 60 mg/kg, or from about 60 mg/kg to about 80 mg/kg, calculated based on the total weight of the animal feed and at least one carrier excipient.

The carrier excipients may include solvent (e.g., water), preservatives, mold inhibitors, and acidity regulators. Acidity regulators may be selected from formic acid, ammonium formate, calcium formate, acetic acid, sodium diacetate, propionic acid, ammonium propionate, sodium propionate, calcium propionate, butyric acid, sodium butyrate, lactic acid, benzoic acid, sodium benzoate, sorbic acid, sodium sorbate, potassium sorbate, fumaric acid, citric acid, potassium citrate, sodium citrate, calcium citrate, tartaric acid, malic acid, phosphoric acid, sodium hydroxide, sodium bicarbonate, potassium chloride, sodium carbonate, calcium acetate, sodium pyrophosphate, sodium tripolyphosphate, sodium hexametaphosphate, sodium metabisulfite, monosodium dihydrogen pyrophosphate, potassium diformate, ammonium chloride, and sodium sulfite.

The animal feed formulation may further comprise at least one additive for animal husbandry, including astaxanthin, oligochitosan, fructooligosaccharides, oligosaccharides, galactooligosaccharides, conjugated linoleic acid, alginate oligosaccharides, curcumin, bile acids, citric acid ketone, plant charcoal black, thirty-seven peptides of dry grass, berberine, red yeast, lutein, β-carotene, chili red, neomethyl hesperidin dihydrochalcone, allicin, monosodium glutamate, disodium 5′-inosinate, disodium 5′-guanylate, and/or tea polyphenols.

Certain embodiments of the present invention are directed to a method of forming a fermentation broth of a microbial strain comprising activating the microbial strain, performing seed cultivation, and preparing a fermentation culture. Certain embodiments of the method further comprise subjecting the fermentation broth to high-temperature inactivation conditions. In certain embodiments of the method, the high-temperature inactivation conditions comprise maintaining the fermentation broth at about 80° C. to about 110° C. for about 0.5 hour to about 2 hours. In certain embodiments of the method, the microbial strain may be a yeast strain, e.g., Saccharomyces cerevisiae or a modified version thereof.

Certain embodiments of the present invention are directed to a method of feeding an animal comprising administering to the animal a feed additive or feed formulation as disclosed herein.

In certain embodiments, the animal is livestock, such as cow, bison, buffalo, hogs, pigs, boar, sheep, goats, rabbits, mink, deer, alligator, snakes, horses, donkeys, chickens, ostrich, turkeys, ducks, geese, quails, game hens, and broiler chickens.

In certain embodiments, the animal is crustaceans, such as lobsters, crabs, shrimp, prawns, and crayfish.

In certain embodiments, the animal is a fish, such as salmon, trout, whitefish, grouper, sea bass, catfish, tuna, carp, and cod.

In certain embodiments, the animal is selected from a grouper, a broiler chicken, and a duck.

In certain embodiments, the administering is performed through liquid administration or by combining with an animal feed formulation.

### DETAILED DESCRIPTION

Salidroside is a bioactive compound found in the root of the Rhodiola rosea plant, having a chemical structure as shown below:

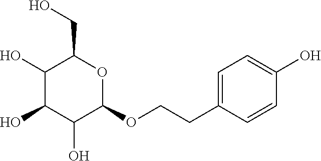 [SMILES: OC[C@H]1O[C@@H](OCCc2ccc(O)cc2)C(O)[C@@H](O)C1O]

Salidroside is the main active ingredient in Rhodiola rosea, and it has a variety of health benefits such as antioxidant, anti-aging, anti-fatigue, anti-depressant, immune-boosting, cardiovascular protection, and anti-altitude sickness effects. The primary method for obtaining salidroside is through plant extraction. However, due to the small size of Rhodiola seeds, their low germination rate, and the long growth cycle of about 7-8 years, coupled with the fact that salidroside content in Rhodiola only accounts for 0.8%-1% of the plant's weight, there is a significant annual demand gap for salidroside. Therefore, it is particularly urgent to develop more efficient alternative production methods.

In the field of animal husbandry, Rhodiola rosea, as a traditional Chinese medicine additive, has been widely used in animal feed. Its rich bioactive components endow the feed with a variety of biological functions, such as anti-inflammatory, antioxidant, hypoglycemic, immune-boosting, and anti-hypoxia effects, which effectively improve the production performance and health status of animals. However, in practical animal breeding, the direct addition of salidroside to feed is extremely rare. More strikingly, there are no cases of direct application of salidroside fermentation liquid in animal husbandry. This indicates that there is still great potential and research space for the application of salidroside and its fermentation liquid in animal husbandry, for which further exploration and development is desirable. The embodiments described herein relate to the addition of salidroside fermentation liquid to feed additives for animal breeding, which has very significant advantages.

Certain embodiments of the present disclosure relate to a fermentation broth comprising salidroside. Certain embodiments further relate to the use of the fermentation broth as a feed additive. For example, in certain embodiments, yeast is used as a feed additive and has a synergistic effect with salidroside in the fermentation broth. This synergistic effect is useful in improving survival rates and body weight in animals, such as layer hens, broiler chickens, meat ducks, crustaceans, shrimp, and groupers.

The use of salidroside extract as a feed additive, for example, in aquaculture, is costly. The present embodiments contemplate direct use of salidroside yeast fermentation broth as a feed additive, which is advantageously more stable and cost-effective than using salidroside extract.

Certain embodiments of the present invention provide a feed additive comprising a fermentation broth of a microbial strain, wherein the microbial strain is selected from the group consisting of yeast strains, Lactobacillus plantarum, Lactobacillus acidophilus, Pediococcus acidilactici, Bacillus coagulans, Bacillus subtilis, Candida tropicalis, and Kluyveromyces marxianus. In at least one embodiment, the microbial strain is Saccharomyces cerevisiae or a modified version thereof, and the fermentation broth comprises salidroside.

In at least one embodiment, Saccharomyces cerevisiae is used as the yeast strain. The fermentation broth of salidroside may be prepared according to the following method: obtaining a strain of yeast (such as the strain described in Chinese Patent Publication No. 110499260 A), activating the strain, performing seed cultivation, and preparing a fermentation culture.

In at least one embodiment, the fermentation and cultivation conditions are: 28-32° C., ventilation rate of 0.8-2.5 vvm (volumes of air per volume of liquid per minute), tank pressure of 0.05-0.08 MPa, and stirring blade speed of 100-800 RPM, dissolved oxygen (DO) not less than 40%, pH control of 5.3-6.0, and cultivation time of 140-170 hours. In an exemplary embodiment, supplementary glucose is added and glucose concentration is controlled in the fermentation broth at 0.1-5 g/L. Yeast powder can be added in batches, and the final concentration after addition is controlled at 6-20 g/L. Sodium hydroxide solution (6 mol/L) may be used for pH regulation.

In at least one embodiment, the fermentation broth of the salidroside yeast strain is used after inactivation. The inactivation conditions can include high-temperature inactivation, preferably maintained from 80-110° C. for 0.5-2 hours, from 90-100° C. for 1.5 hours, from 95-105° C. for 1.5 hours, or from 100-105° C. for 1 hour.

To ensure the stability of the salidroside fermentation liquid, it may be used after being treated with an acidity regulator. In at least one embodiment, the acidity regulator is selected from formic acid, ammonium formate, calcium formate, acetic acid, sodium diacetate, propionic acid, ammonium propionate, sodium propionate, calcium propionate, butyric acid, sodium butyrate, lactic acid, benzoic acid, sodium benzoate, sorbic acid, sodium sorbate, potassium sorbate, fumaric acid, citric acid, potassium citrate, sodium citrate, calcium citrate, tartaric acid, malic acid, phosphoric acid, sodium hydroxide, sodium bicarbonate, potassium chloride, sodium carbonate, calcium acetate, sodium pyrophosphate, sodium tripolyphosphate, sodium hexametaphosphate, sodium metabisulfite, monosodium dihydrogen pyrophosphate, potassium diformate, ammonium chloride, sodium sulfite, and combinations thereof.

In at least one embodiment, the pH of the salidroside fermentation broth is adjusted to a range of about 2.5 to about 3.5, including specific values such as about pH 2.5, about pH 2.6, about pH 2.7, about pH 2.8, about pH 2.9, about pH 3.0, about pH 3.1, about pH 3.2, about pH 3.3, about pH 3.4, about pH 3.5, or within any range defined by and inclusive of these points (e.g., about pH 3.0 to about pH 3.4), which ensures greater stability. Preferably, the pH adjustment of the salidroside fermentation liquid can be achieved using an acidity regulator.

In at least one embodiment, an animal feed formulation comprises: a feed additive of salidroside fermentation; and at least one carrier excipient.

In at least one embodiment, an animal feed formulation comprises salidroside present from about 10 mg/kg to about 100 mg/kg, calculated based on total weight of the animal feed formulation and at least one carrier excipient. An animal feed formulation may comprise salidroside present at about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or within any range defined by and inclusive of these points (e.g., about 45 mg/kg to about 90 mg/kg). Preferably, the salidroside is present from about 20 mg/kg to about 60 mg/kg.

In at least one embodiment, the animal feed formulation further comprises at least one additive for animal husbandry selected from astaxanthin, oligochitosan, fructooligosaccharides, oligosaccharides, galactooligosaccharides, conjugated linoleic acid, alginate oligosaccharides, curcumin, bile acids, citric acid ketone, plant charcoal black, thirty-seven peptides of dry grass, berberine, red yeast, lutein, β-carotene, chili red, neomethyl hesperidin dihydrochalcone, allicin, monosodium glutamate, disodium 5′-inosinate, disodium 5′-guanylate, tea polyphenols, and combinations thereof.

In at least one embodiment, a method of feeding an animal comprises administering to the animal a feed additive or animal feed formulation.

Embodiments of the present disclosure contemplate the application of the fermentation broth of Rhodiola rosea yeast strain in animal husbandry, such as poultry, livestock, and aquaculture (e.g., raising fish and/or shrimp). The broth may be combined with other ingredients pre-sale, and/or may include instructions on how to combine the broth with other components to produce a desired feed formulation.

Advantages of the embodiments described herein include, but are not limited to: (1) improving the disease resistance and reducing the mortality rate of fish and shrimp in aquaculture, as increasing the weight and fullness of aquatic products can improve the profitability of fish farming; (2) animal husbandry, and in particular improving the survival rate of animals; and (3) providing a comprehensive feed additive that can improve the immune function of animals and can be produced at large scale and low cost.

As used herein, “fermentation broth” refers to an aqueous solution/suspension comprising whole cells, water, media, biomolecules excreted by the whole cells, and other constituents within a fermentation vessel.

Also as used herein, “organ-body ratio” refers to the ratio of the weight of organ in an experimental animal to its body weight.

Also as used herein, “fat fullness” refers to the ratio of fish body weight to the cube of fish body length, which is an indicator that reflects the degree of fish fat and growth.

Also as used herein, “feed conversion ratio” refers to the amount of feed consumed per kilogram of weight gain for livestock and poultry raised.

Also as used herein, “chemical stability” refers to the ability of the feed additive, fermentation broth, or relevant composition to retain its intended chemical composition and potency over a specified period of storage under one or more defined conditions. Further, “chemical stability” implies the absence of significant formation of degradation products, preservation of physical appearance (e.g., color, clarity, absence of precipitation), and maintenance of functional properties relevant to animal husbandry applications. Unless otherwise indicated, storage conditions are understood to be at ambient temperature (approximately 20-25° C.), protected from direct sunlight, and under typical humidity levels encountered in feed storage environments.

Also as used herein, “about” refers to a value or range that is close to, approximately, or nearly the stated numerical value, allowing for minor variations that do not materially affect the basic and novel characteristics of the invention. Unless otherwise specified, the term “about” is intended to encompass values within a reasonable range of the stated value, typically within +10%, but may vary depending on the context and precision of measurement relevant to the field.

The following exemplary embodiments are now described.

Embodiment 1: A feed additive comprising a fermentation broth of a microbial strain.

Embodiment 2: The feed additive of Embodiment 1, wherein the microbial strain is a yeast strain.

Embodiment 3: The feed additive of Embodiment 2, wherein the yeast strain is a Saccharomyces cerevisiae or a modified version thereof.

Embodiment 4: The feed additive of any of Embodiments 1-3, wherein the feed additive maintains at least 95% chemical stability after about six months storage at room temperature or approximately 20-25° C.

Embodiment 5: The feed additive of any of Embodiments 1-4, wherein the fermentation broth comprises salidroside.

Embodiment 6: The feed additive of Embodiment 2, wherein the fermentation broth comprises whole yeast cells, water, media, and biomolecules excreted by the yeast strain.

Embodiment 7: The feed additive of Embodiment 1, wherein the fermentation broth is produced by a process comprising activating a yeast strain; performing seed cultivation; and performing fermentation under controlled conditions of temperature, aeration, pressure, stirring speed, dissolved oxygen, and pH.

Embodiment 8: The feed additive of Embodiment 1, wherein the fermentation broth is subjected to high-temperature inactivation conditions, comprising maintaining the fermentation broth at a temperature of about 80° C. to about 120° C. for about 0.5 hour to about 2 hours.

Embodiment 9: An animal feed formulation comprising: the feed additive of any of Embodiments 1-5; and at least one carrier excipient.

Embodiment 10: An animal feed formulation comprising salidroside present from about 10 mg/kg to about 100 mg/kg, calculated based on total weight of the animal feed formulation and at least one carrier excipient.

Embodiment 11: The animal feed formulation of Embodiment 10, wherein the salidroside is present from about 20 mg/kg to about 60 mg/kg.

Embodiment 12: The animal feed formulation of either Embodiment 10 or Embodiment 11, further comprising at least one additive for animal husbandry selected from astaxanthin, oligochitosan, fructooligosaccharides, oligosaccharides, galactooligosaccharides, conjugated linoleic acid, alginate oligosaccharides, curcumin, bile acids, citric acid ketone, plant charcoal black, thirty-seven peptides of dry grass, berberine, red yeast, lutein, β-carotene, chili red, neomethyl hesperidin dihydrochalcone, allicin, monosodium glutamate, disodium 5′-inosinate, disodium 5′-guanylate, tea polyphenols, and combinations thereof.

Embodiment 13: The animal feed formulation of any one of Embodiments 10-12, further comprising an acidity regulator selected from formic acid, ammonium formate, calcium formate, acetic acid, sodium diacetate, propionic acid, ammonium propionate, sodium propionate, calcium propionate, butyric acid, sodium butyrate, lactic acid, benzoic acid, sodium benzoate, sorbic acid, sodium sorbate, potassium sorbate, fumaric acid, citric acid, potassium citrate, sodium citrate, calcium citrate, tartaric acid, malic acid, phosphoric acid, sodium hydroxide, sodium bicarbonate, potassium chloride, sodium carbonate, calcium acetate, sodium pyrophosphate, sodium tripolyphosphate, sodium hexametaphosphate, sodium metabisulfite, monosodium dihydrogen pyrophosphate, potassium diformate, ammonium chloride, sodium sulfite, and combinations thereof.

Embodiment 14: A method of forming a fermentation broth of a microbial strain comprising: activating the microbial strain; performing seed cultivation; and performing fermentation.

Embodiment 15: The method of Embodiment 14, further comprising: subjecting the fermentation broth to high-temperature inactivation conditions.

Embodiment 16: The method of Embodiment 15, wherein the high-temperature inactivation conditions comprise maintaining the fermentation broth at about 80° C. to about 110° C. for about 1 hour to about 2 hours.

Embodiment 17: The method of Embodiment 14, wherein the pH of the salidroside fermentation broth is adjusted to a range of about 2.5 to about 3.5.

Embodiment 18: The method of any of Embodiments 14-17, wherein the microbial strain is a yeast strain.

Embodiment 19: The method of Embodiment 18, wherein the yeast strain is a Saccharomyces cerevisiae or a modified version thereof.

Embodiment 20: The method of any of Embodiments 14-19, wherein the fermentation broth comprises salidroside.

Embodiment 21: A method of feeding an animal comprising: administering to the animal a feed additive or feed formulation of any of Embodiments 1-13.

Embodiment 22: The method of Embodiment 21, wherein the animal is livestock.

Embodiment 23: The method of Embodiment 22, wherein the livestock is selected from cow, bison, buffalo, hogs, pigs, boar, sheep, goats, rabbits, mink, deer, alligator, snakes, horses, donkeys, chickens, ostrich, turkeys, ducks, geese, quails, game hens, and broiler chicken.

Embodiment 24: The method of Embodiment 21, wherein the animal is fish or crustaceans.

Embodiment 25: The method of Embodiment 24, wherein the crustaceans are selected from lobsters, crabs, shrimp, prawns and crayfish.

Embodiment 26: The method of Embodiment 24, wherein the fish are selected from salmon, trout, whitefish, grouper, sea bass, catfish, tuna, carp and cod.

Embodiment 27: The method of Embodiment 21, wherein the animal is selected from a grouper, a broiler chicken, and a duck.

Embodiment 28: The method of any of Embodiments 21-27, wherein the administering is performed through liquid administration or by combination with an animal feed formulation.

### ILLUSTRATIVE EXAMPLES

The following examples are set forth to assist in understanding the disclosed embodiments and should not be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

The examples relate to obtaining a Rhodiola glycoside fermentation liquid. The culture medium used for yeast production of salidroside includes 20 g/L glucose, 20 g/L peptone, and 10 g/L yeast powder. The production strain of salidroside is derived as described in Chinese Patent Publication No. 110499260 A.

### Example 1: Efficient Fermentation of Salidroside by Brewing Yeast Engineering Bacteria in a 100 L Fermentation Tank

The following protocol was used for fermentation of salidroside, including the steps of strain activation, seed cultivation, and preparing the fermentation culture.

(1) Strain activation: Inoculate the Saccharomyces cerevisiae engineering strain into YPD agar and activate it at 30° C. for 44 hours.

(2) First level seed cultivation: Select a single colony of brewing yeast engineering bacteria from the flat plate culture medium at the end of the activation step (1), inoculate it into a test tube with a liquid volume of 5 mL, a total of 20 tubes, and cultivate under the following conditions: shaking table culture, temperature of 30° C., 220 RPM, and cultivation time of 22 hours.

(3) Secondary seed cultivation: Transfer 100 mL of the cultivated primary seed solution to a 10 L fermentation tank with a liquid volume of 5 L, with an inoculation amount of 2%. The cultivation conditions are: temperature of 30° C., initial pH of 6.35, aeration rate of 1.5 vvm, constant tank pressure of 0.05 MPa, and constant stirring speed of 300 RPM. When the culture reaches 7 hours, the pH drops to 5.78, and the pH automatic control is activated to maintain it at 5.80 until the end of the culture. When the cultivation reaches 8.5 hours, the cultivation is terminated and transferred to a 100 L fermentation tank for fermentation and cultivation.

(4) Fermentation culture: Transfer 5 L of secondary seed liquid into a 100 L fermentation tank with a liquid volume of 50 L, inoculate 10%, and culture conditions: temperature of 30° C., initial stirring speed of 200 RPM, initial pH of 6.30, initial aeration rate of 0.8 vvm, which can be increased to no more than 1.2 vvm as needed. The initial tank pressure is 0.05 MPa, and it can be increased to no more than 0.08 MPa as needed. When cultured for 6 hours, the pH drops to 5.78, and automatic pH control is activated to maintain it at 5.80 until the end of fermentation. When cultured for 11 hours, the DO is 35%. Increase the stirring speed, with an upper limit of no more than 600 RPM, and maintain the DO at no less than 40% until fermentation is complete. When the cultivation reaches 8 hours, glucose is added in a continuous flow and yeast powder is added at the same time. The subsequent glucose addition is performed in a continuous flow manner, while yeast powder is added in batches with an interval of 8 hours, for a total of 12 yeast powder additions. Continue cultivation until 140 hours and stop adding glucose until zero residual sugar level is reached at 144 hours, ending fermentation. At this point, the content of salidroside is 31.38 g/L.

The glucose concentration used for flow addition was 800 g/L, the concentration of yeast powder used for batch supplementation was 400 g/L, and the concentration of sodium hydroxide used for pH self-control was 6 mol/L.

(4) Fermentation broth for post-treatment: The fermentation broth obtained above was kept at 80-120° C. for 1-2 hours to obtain a feed additive containing salidroside.

### Example 2: Efficient Fermentation of Salidroside by Brewing Yeast Engineering Bacteria in a 5 T Fermentation Tank

The following protocol was used for fermentation of salidroside, including the steps of strain activation, seed cultivation, preparing the fermentation culture.

(1) Strain activation: Inoculate the Saccharomyces cerevisiae engineering strain into YPD agar and activate it at 30° C. for 44 hours.

(2) First level seed cultivation: Select a single colony of brewing yeast engineering bacteria from the flat plate culture medium at the end of the activation step (1), inoculate it into a flask with a liquid volume of 25 mL, a total of 30 flasks, and cultivate under the following conditions: shaking table culture, temperature of 30° C., 220 RPM, and cultivation time of 22 hours.

(3) Secondary seed cultivation: Transfer 500 mL of the cultivated primary seed solution to a 50 L fermentation tank with a liquid volume of 25 L, with an inoculation amount of 2%. The cultivation conditions are: temperature of 30° C., initial pH of 6.45, aeration rate of 1.5 vvm, keep fermentation tank pressure of 0.05 MPa, and constant stirring speed of 300 RPM. When the culture reaches 7.4 hours, the pH drops to 5.78, and the pH automatic control is activated to maintain it at 5.80 until the end of the culture. End the cultivation when it reaches 8.5 hours.

(4) Third level seed cultivation: Transfer 25 L of secondary seed solution to a 500 L fermentation tank with a liquid volume of 250 L, inoculate 10%, and cultivate under the following conditions: temperature of 30° C., initial pH of 6.30, aeration rate of 1 vvm, constant tank pressure of 0.05 MPa, and constant stirring speed of 300 RPM. When the pH drops to 5.78 after 5 hours of cultivation, the pH automatic control is activated and maintained at 5.80 until the end of cultivation. End the cultivation at 5.5 hours.

(5) Fermentation culture: Transfer 250 L of tertiary seed liquid into a 5,000 L fermentation tank with a liquid capacity of 2,500 L, inoculate at 10%, and cultivate under the following conditions: temperature of 30° C., initial stirring speed of 100 RPM, initial pH of 6.20, initial aeration rate of 0.8 vvm, which can be increased to no more than 1.7 vvm as needed. The initial tank pressure is 0.05 MPa, and it can be increased to no more than 0.08 MPa as needed. When the culture reaches 4 hours, the pH drops to 5.78, and the pH automatic control is turned on, maintaining it at 5.80 until the end of fermentation. When the cultivation reaches 12 hours, the DO is 39%. Increase the stirring speed, with the upper limit of the speed not exceeding 240 RPM, and maintain the DO at not less than 50% until the end of fermentation. When the culture reaches 10 hours, glucose supplementation is started and yeast powder is added simultaneously. The subsequent glucose supplementation method is flow supplementation, and yeast powder supplementation method is batch supplementation with an interval of 8 hours, for a total of 12 supplements. Continue cultivation until 154 hours and stop adding glucose until zero residual sugar level is reached at 158 hours, ending fermentation. At this time, the content of salidroside is 30.36 g/L.

The glucose concentration used for flow addition was 800 g/L, the concentration of yeast powder used for batch supplementation was 400 g/L, and the concentration of sodium hydroxide used for pH self-control was 6 mol/L.

(6) Fermentation broth for post-treatment: The fermentation broth obtained above was kept at 80-120° C. for 1-2 hours to obtain a feed additive containing salidroside.

### Example 3: Stability of Salidroside Fermentation Broth

In this protocol, the pH of the fermentation broth obtained in Example 1 or Example 2 was adjusted to approximately 3 in a sterile environment, and then its stability was assessed in a high-temperature and high-humidity environment.

The testing conditions were 40° C., 75% RH, and tests were carried out on the 7th, 15th, 30th and 60th day respectively. The measured parameters included the content of salidroside detected by high-performance liquid chromatography (HPLC), microbial detection, and pH value to evaluate degradation degree, with the results summarized in Table 1.

TABLE 1
Stability of salidroside fermentation broth
under high temperature and humidity

| Items | Day 0 | Day 7 | Day 15 | Day 30 | Day 60 |
|---|---|---|---|---|---|
| Salidroside content (%) | 2.14 | 2.07 | 2.06 | 2.04 | 2.05 |
| Salidroside mass (g) | 1.098 | 1.062 | 1.063 | 1.050 | 1.055 |
| Microbial (CFU/mL) | 0 | 0 | 0 | 0 | 0 |
| pH | 2.80 | 2.90 | 2.95 | 2.87 | 3.00 |

### Example 4: Fermentation Broth of Salidroside Used for the Breeding of Layer Hens

The experiment was divided into groups A, B, C, and D, with 3 parallel groups in each group and 6 laying hens in each parallel group.

Feeding plan: Group A is the control group, without adding salidroside fermentation broth. B. C and D groups were added with 10 mg salidroside equivalent/kg feed, 20 mg salidroside equivalent/kg feed, and 40 mg salidroside equivalent/kg feed, respectively, to the salidroside fermentation broth. Spray the fermentation broth onto daily feed and dry it before use.

Breeding cycle: two month feeding cycle, various growth indicators of laying hens were tested, and the results are summarized in Table 2, which includes standard error of the mean (SEM) and P-value for each data point. It is noted that different lowercase letters on the shoulder of the same column indicate significant differences (P<0.05), while the same lowercase letters or no letters indicate no significant differences (P>0.05).

TABLE 2
Effect of Rhodiola rosea glycoside on the growth performance of laying hens

| | | Group | | | | | P- |
|---|---|---|---|---|---|---|---|
| Stage | Items | A | B | C | D | SEM | value |
| Week | Egg production (%) | 65.08 | 87.41 | 79.88 | 83.92 | 0.04 | 0.11 |
| 1-4 | Average daily feed | 129.68 | 131.46 | 130.57 | 135.19 | 0.93 | 0.16 |
| | intake (g) | | | | | | |
| | Average egg | 63.44^{c} | 64.88^{bc} | 65.61^{b} | 68.84^{a} | 0.48 | <0.001 |
| | weight (g) | | | | | | |
| | Feed egg ratio | 2.84 | 2.73 | 2.73 | 2.61 | 0.09 | 0.87 |
| | Egg breakage | 1.99 | 1.79 | 0.30 | 0.28 | 0.004 | 0.19 |
| | rate (%) | | | | | | |
| Week | Egg production (%) | 49.57^{b} | 83.86^{a} | 83.91^{a} | 87.99^{a} | 0.06 | 0.02 |
| 5-8 | Average daily feed | 134.68 | 134.18 | 131.74 | 135.55 | 1.80 | 0.91 |
| | intake (g) | | | | | | |
| | Average egg | 62.91^{c} | 64.32^{bc} | 66.13^{b} | 68.56^{a} | 0.58 | <0.001 |
| | weight (g) | | | | | | |
| | Feed egg ratio | 4.42^{a} | 2.79^{b} | 2.66^{b} | 2.38^{b} | 0.29 | 0.04 |
| | Egg breakage | 4.39 | 2.81 | 1.74 | 0.86 | 0.007 | 0.38 |
| | rate (%) | | | | | | |
| Week | Egg production (%) | 59.07^{b} | 85.26^{a} | 84.77^{a} | 89.24^{a} | 0.04 | 0.02 |
| 1-8 | Average daily feed | 132.18 | 132.81 | 131.15 | 135.37 | 1.21 | 0.67 |
| | intake (g) | | | | | | |
| | Average egg | 63.18^{d} | 64.60^{c} | 65.87^{b} | 68.70^{a} | 0.46 | <0.001 |
| | weight (g) | | | | | | |
| | Feed egg ratio | 3.37 | 2.76 | 2.69 | 2.51 | 0.14 | 0.14 |
| | Egg breakage | 2.84 | 2.25 | 0.89 | 0.57 | 0.46 | 0.25 |
| | rate (%) | | | | | | |

As shown in Table 2, adding appropriate concentrations of salidroside fermentation broth to feed can effectively improve egg production rate, average egg weight, reduce feed egg ratio, and egg breakage rate.

Eggs produced by four groups of laying hens were collected for egg quality analysis, and the analysis results are shown in Table 3. It is noted that different lowercase letters on the shoulder of the same column indicate significant differences (P<0.05), while the same lowercase letters or no letters indicate no significant differences (P>0.05).

TABLE 3
The effect of Rhodiola glycosides on the quality of eggs

| | | Group | | | | | P- |
|---|---|---|---|---|---|---|---|
| Stage | Items | A | B | C | D | SEM | value |
| Second | Egg weight (g) | 63.41^{c} | 64.79^{bc} | 65.42^{b} | 68.77^{a} | 0.37 | <0.001 |
| week | Shape index (%) | 1.37 | 1.38 | 1.36 | 1.36 | 0.004 | 0.34 |
| | Eggshell | 34.67 | 35.85 | 35.12 | 36.06 | 0.46 | 0.70 |
| | strength (N/cm²) | | | | | | |
| | Protein | 7.89^{c} | 8.50^{ab} | 8.04^{bc} | 8.87^{a} | 0.10 | <0.001 |
| | height (mm) | | | | | | |
| | Yolk color | 6.96^{b} | 7.29^{b} | 7.13^{b} | 7.86^{a} | 0.09 | 0.002 |
| | Eggshell | 0.39 | 0.40 | 0.40 | 0.39 | 0.003 | 0.07 |
| | thickness (mm) | | | | | | |
| | Haugh unit | 87.90 | 88.98 | 87.83 | 91.19 | 0.52 | 0.09 |
| Fourth | Egg weight (g) | 64.62^{b} | 64.53^{b} | 66.17^{ab} | 68.64^{a} | 0.48 | 0.006 |
| week | Shape index (%) | 1.36 | 1.39 | 1.37 | 1.37 | 0.005 | 0.31 |
| | Eggshell | 29.10^{b} | 27.64^{b} | 33.21^{a} | 33.13^{a} | 0.73 | 0.008 |
| | strength (N/cm²) | | | | | | |
| | Protein | 8.46^{c} | 9.16^{b} | 9.10^{b} | 9.61^{a} | 0.09 | <0.001 |
| | height (mm) | | | | | | |
| | Yolk color | 6.78^{b} | 7.78^{a} | 7.72^{a} | 7.05^{b} | 0.10 | <0.001 |
| | Eggshell | 0.36^{a} | 0.35^{b} | 0.37^{a} | 0.36^{a} | 0.002 | 0.002 |
| | thickness (mm) | | | | | | |
| | Haugh unit | 89.68^{b} | 94.27^{a} | 93.51^{a} | 95.20^{a} | 0.46 | <0.001 |
| Sixth | Egg weight (g) | 63.47^{c} | 65.94^{b} | 66.54^{b} | 69.63^{a} | 0.50 | <0.001 |
| week | Shape index (%) | 1.43 | 1.41 | 1.41 | 1.40 | 0.004 | 0.15 |
| | Eggshell | 32.23^{c} | 37.42^{a} | 36.69^{ab} | 33.29^{bc} | 0.74 | 0.005 |
| | strength (N/cm²) | | | | | | |
| | Protein | 7.06^{c} | 7.53^{b} | 7.29^{bc} | 8.29^{a} | 0.09 | <0.001 |
| | height (mm) | | | | | | |
| | Yolk color | 6.78 | 6.80 | 6.74 | 6.85 | 0.09 | 0.98 |
| | Eggshell | 0.37^{b} | 0.38^{b} | 0.38^{b} | 0.40^{a} | 0.003 | <0.001 |
| | thickness (mm) | | | | | | |
| | Haugh unit | 83.34^{b} | 85.31^{b} | 83.83^{b} | 88.42^{a} | 0.53 | 0.002 |
| Eighth | Egg weight (g) | 62.56^{b} | 64.34^{b} | 68.62^{a} | 70.07^{a} | 0.58 | <0.001 |
| week | Shape index (%) | 1.40 | 1.42 | 1.40 | 1.39 | 0.006 | 0.27 |
| | Eggshell | 32.18^{ab} | 35.83^{a} | 29.98^{b} | 35.71^{a} | 0.84 | 0.04 |
| | strength (N/cm²) | | | | | | |
| | Protein | 6.38^{b} | 7.00^{a} | 7.06^{a} | 7.26^{a} | 0.10 | 0.01 |
| | height (mm) | | | | | | |
| | Yolk color | 6.58^{b} | 6.67^{b} | 6.56^{b} | 7.42^{a} | 0.10 | <0.001 |
| | Eggshell | 0.38^{b} | 0.39^{ab} | 0.41^{a} | 0.41^{a} | 0.003 | 0.04 |
| | thickness (mm) | | | | | | |
| | Haugh unit | 78.53^{b} | 82.32^{a} | 80.50^{ab} | 83.03^{a} | 0.53 | 0.002 |

From Table 3, it can be seen that salidroside can significantly increase protein height and improve Haugh units.

Blood samples were collected from four groups of laying hens after 2 months of feeding for immune related indicators and analyzed. The results are shown in Table 4. It is noted that different lowercase letters on the shoulder of the same column indicate significant differences (P<0.05), while the same lowercase letters or no letters indicate no significant differences (P>0.05).

TABLE 4
Effects of Rhodiola rosea glycoside
on immune indicators of laying hens

| | Group | | | | | P- |
|---|---|---|---|---|---|---|
| Items | A | B | C | D | SEM | value |
| IgA(ng/mL) | 40.04^{b} | 43.59^{a} | 44.09^{a} | 43.86^{a} | 0.51 | <0.001 |
| IgG(μg/mL) | 2.55^{b} | 6.55^{a} | 6.60^{a} | 7.60^{a} | 0.60 | <0.001 |
| IgM(μg/mL) | 3.52 | 4.36 | 3.96 | 5.36 | 0.27 | 0.07 |
| IL-6(pg/mL) | 52.20^{a} | 38.87^{ab} | 23.88^{b} | 22.41^{b} | 4.30 | 0.01 |
| IL-10(pg/mL) | 45.03^{b} | 92.70^{a} | 85.44^{a} | 102.81^{a} | 7.35 | 0.003 |
| IL-1β(pg/mL) | 53.04^{a} | 50.24^{ab} | 46.77^{b} | 45.45^{b} | 1.11 | 0.03 |

As shown, it can be seen that salidroside can significantly increase the levels of IgA, IgG, and IgM in laying hens, enhancing their immunity, with Group D showing particularly significant effects. Meanwhile, salidroside can significantly reduce the expression levels of inflammatory factors IL-6 and IL-1β, and significantly increase the expression level of anti-inflammatory factor IL-10, helping the body resist inflammation, with group D showing the best performance.

Blood samples were collected from four groups of laying hens for biochemical testing and analyzed. The results are shown in Table 5. It is noted that different lowercase letters on the shoulder of the same column indicate significant differences (P<0.05), while the same lowercase letters or no letters indicate no significant differences (P>0.05).

TABLE 5
The effect of salidroside on blood biochemical indicators of laying hens

| | Group | | | | | P- |
|---|---|---|---|---|---|---|
| Items | A | B | C | D | SEM | value |
| Total Protein TP (g/L) | 53.83 | 54.72 | 54.01 | 54.30 | 0.19 | 0.42 |
| Albumin ALB (g/L) | 21.06 | 20.69 | 20.81 | 20.86 | 0.10 | 0.68 |
| Alanine | 19.57^{a} | 16.69^{b} | 16.20^{b} | 15.47^{b} | 0.58 | 0.02 |
| Aminotransferase ALT (U/L) | | | | | | |
| Aspartate | 277.71^{a} | 264.93^{b} | 244.98^{c} | 239.76^{d} | 3.96 | <0.001 |
| Aminotransferase AST (U/L) | | | | | | |
| Creatinine CRE (μmol/L) | 4.79^{a} | 3.79^{b} | 4.01^{ab} | 3.34^{b} | 0.19 | 0.02 |
| Blood Urea Nitrogen | 5.26^{a} | 1.92^{c} | 2.51^{c} | 3.96^{b} | 0.42 | <0.001 |
| BUN (mg/dL) | | | | | | |
| Glucose GLU (mmol/L) | 11.74^{a} | 10.69^{b} | 10.53^{b} | 10.73^{b} | 0.12 | <0.001 |
| Cholesterol CHO (mmol/L) | 2.59^{a} | 1.87^{b} | 1.79^{b} | 2.08^{ab} | 0.11 | 0.02 |
| Triglycerides TG (mmol/L) | 13.55^{a} | 5.95^{d} | 7.61^{c} | 10.19^{b} | 0.81 | <0.001 |

As shown, Rhodiola rosea glycoside can significantly reduce the activity of alanine aminotransferase and aspartate aminotransferase, and has the effect of protecting the liver. Meanwhile, salidroside can reduce creatinine and urea nitrogen, protecting the kidney health of laying hens. Rhodiola rosea glycoside also has significant hypoglycemic and lipid-lowering effects, ensuring the health of the body.

Blood and ovarian tissue samples were collected from four groups of laying hens to detect relevant hormone indicators and analyzed. The results are shown in Table 6. It is noted that different lowercase letters on the shoulder of the same column indicate significant differences (P<0.05), while the same lowercase letters or no letters indicate no significant differences (P>0.05).

TABLE 6
The effect of salidroside on hormonal indicators in laying hens

| | Group | | | | | P- |
|---|---|---|---|---|---|---|
| Items | A | B | C | D | SEM | value |
| Estrogen E2/(pg/mL) | 218.10^{c} | 369.29^{b} | 381.29^{b} | 424.73^{a} | 17.54 | <0.001 |
| Follicle-Stimulating | 6.83^{b} | 10.80^{a} | 10.26^{a} | 11.28^{a} | 0.36 | <0.001 |
| Hormone FSH/(mIU/mL) | | | | | | |
| Gonadotropin- | 60.00^{c} | 75.60^{b} | 76.69^{b} | 82.74^{a} | 1.98 | <0.001 |
| Releasing Hormone | | | | | | |
| GnRH/(mIU/ml) | | | | | | |
| Luteinizing Hormone | 105.38^{c} | 133.01^{b} | 143.72^{a} | 151.15^{a} | 4.09 | <0.001 |
| LH/(ng/ml) | | | | | | |
| Progesterone | 7.82^{c} | 12.20^{b} | 12.51^{ab} | 13.34^{a} | 0.43 | <0.001 |
| PROG/(ng/ml) | | | | | | |
| Cortisol | 503.84^{a} | 423.94^{b} | 415.52^{b} | 372.00^{c} | 12.33 | <0.001 |
| CORT/(pg/mL) | | | | | | |

From Table 6, it can be seen that salidroside can significantly increase the levels of estradiol, follicle stimulating hormone, gonadotropin-releasing hormone, luteinizing hormone, and progesterone, and has a significant promoting effect on the development of the reproductive system in laying hens, with Group D showing the most significant effect. Rhodiola rosea glycoside can significantly reduce cortisol levels and alleviate stress symptoms in laying hens.

### Example 5: Fermentation Broth of Salidroside Used for the Breeding of Meat Ducks

The experiment was divided into two groups, A and B, with 3 parallel groups in each group and 10 meat ducks in each parallel group.

Feeding plan: Group A was the control group, without adding salidroside fermentation broth. Group B had 20 mg salidroside equivalent/kg feed amount of salidroside fermentation broth. Fermented liquid was sprayed onto the daily feed and dried before use.

Breeding cycle: seven weeks. After the breeding was completed, the slaughter index was tested, and the results are summarized in Table 7.

TABLE 7
Effect of Rhodiola rosea glycoside on slaughter index of meat ducks

| Four weeks slaughter index | | | Seven weeks slaughter index | | |
|---|---|---|---|---|---|
| | | Rhodiola | | | Rhodiola |
| | | glycoside | | | glycoside |
| | Control | group | | Control | group |
| Heart (g) | 7.50 ± 0.63 | 8.67 ± 0.19 | Heart (g) | 16.64 ± 1.68 | 16.81 ± 0.32 |
| Liver (g) | 35.96 ± 2.50 | 35.61 ± 1.46 | Liver (g) | 45.63 ± 6.93 | 42.27 ± 1.93 |
| Spleen (g) | 1.32 ± 0.11 | 1.46 ± 0.11 | Spleen (g) | 2.40 ± 0.50 | 2.10 ± 0.48 |
| Lung (g) | 10.50 ± 0.55 | 11.04 ± 0.21 | Lung (g) | 25.42 ± 4.25 | 25.24 ± 4.24 |
| Kidney (g) | 13.25 ± 0.97 | 15.47 ± 0.57 | Kidney (g) | 18.75 ± 3.35 | 18.63 ± 3.24 |
| Thymus (g) | 6.50 ± 0.48 | 6.57 ± 0.47 | Thymus (g) | 9.22 ± 4.72 | 10.66 ± 1.68 |
| Gizzard (g) | 33.24 ± 1.10 | 40.22 ± 1.64 | Gizzard (g) | 50.92 ± 5.06 | 54.75 ± 9.59 |
| Chest muscles (g) | 46.93 ± 3.00 | 44.78 ± 2.00 | Chest muscles (g) | 263.54 ± 50.24 | 277.64 ± 26.26 |
| Leg muscles (g) | 167.62 ± 6.05 | 164.08 ± 8.51 | Leg muscles (g) | 194.04 ± 28.17 | 176.41 ± 21.54 |
| Tibia length (cm) | 7.53 ± 0.13 | 7.55 ± 0.12 | Tibia length (cm) | 7.84 ± 0.51 | 8.01 ± 0.22 |
| Tibia circumference (cm) | 4.75 ± 0.10 | 4.93 ± 0.08 | Tibia circumference (cm) | 5.39 ± 0.67 | 5.40 ± 0.09 |
| Carcass weight (g) | 1210.68 ± 22.08 | 1264.86 ± 35.05 | Carcass weight (g) | 2479.07 ± 225.00 | 2504.21 ± 146.10 |
| | | | Right Heart Index RV/TV | 0.42 ± 0.11 | 0.27 ± 0.05 |

Adding 20 mg salidroside equivalent fermentation broth to every kg of feed did not significantly increase the weight gain of meat ducks. The significant decrease in right heart index indicates that salidroside has a good effect on right ventricular hypertrophy caused by hypoxia.

### Example 6: Rhodiola Glycoside Fermentation Broth for Broiler Farming

The experiment was divided into three groups: A, B, and C. Group A was the control group, with 12,200 broilers. Group B was an experimental group with 17,000 broilers, and Group C was an experimental group with 12,200 broilers.

Feeding plan: Group A was the control group, without adding salidroside fermentation broth. Group B had 20 mg salidroside equivalent/kg feed of salidroside fermentation broth, and Group C had 60 mg salidroside equivalent/kg feed of salidroside fermentation broth. Fermentation broth was fed through drinking water. The breeding cycle was six weeks. Results are summarized in Table 8.

TABLE 8
The effect of salidroside fermentation broth on
the weight and survival rate of broiler chickens

| | | | Average | | |
|---|---|---|---|---|---|
| | Net | | weight per | Survival | Feed |
| | weight | Number of | chicken | rate | conversion |
| | (kg) | chickens | (kg/chicken) | (%) | ratio |
| A (control) | 25316 | 8896 | 2.85 | 72.33 | 1.48 |
| B | 42994 | 14876 | 2.89 | 87.51 | 1.46 |
| C | 31882 | 11104 | 2.87 | 90.28 | 1.47 |

There was no significant difference in weight and feed to gain ratio between the experimental group and the control group, but there was a significant difference in survival rate. This experiment shows similar performance to the grouper farming experiment, which can increase the disease resistance and survival rate of the fed animals.

In the process of broiler farming, the experimental group fed with salidroside fermentation broth exhibited improved recovery of respiratory tract infections in broiler chickens. When animals suffer from respiratory diseases and have insufficient oxygen intake, Rhodiola rosea can improve oxygen utilization efficiency. This finding is consistent with the results of the duck farming experiments.

### Example 7: Fermentation Broth of Salidroside Used for the Breeding of Grouper

The experiment was divided into groups A, B, C, and D, with 3 parallel groups of groupers in each group and 30 groupers in each parallel group.

Feeding plan: Group A was the control group, without adding salidroside fermentation broth. B, C and D groups were added with 10 mg salidroside equivalent/kg feed, 20 mg salidroside equivalent/kg feed, and 40 mg salidroside equivalent/kg feed of salidroside fermentation broth, respectively. Fermented liquid was sprayed onto the daily feed and dried before use.

Breeding cycle: For two months the survival rate and various growth indicators of grouper were tested, and the results are summarized in Table 9. It is noted that different lowercase letters on the shoulder of the same column indicate significant differences (P<0.05), while the same lowercase letters or no letters indicate no significant differences (P>0.05).

TABLE 9
Effects of salidroside on growth performance indexes of grouper

| | | Specific | | | Feed | |
|---|---|---|---|---|---|---|
| Growth | Survival | growth | Weight | Fat | conversion | Organ- |
| indicators | rate (%) | rate (%) | gain (%) | fullness | ratio | body ratio |
| A (control) | 83.33 ± 0^{b} | 1.1 ± 0.04^{b} | 75.44 ± 3.47^{c} | 3.07 ± 0.10^{b} | 1.12 ± 0.1^{b} | 10.42 ± 1.07^{b} |
| B | 91.11 ± 1.92^{a} | 1.16 ± 0.05^{a} | 91.03 ± 5.73^{a} | 4.41 ± 0.59^{a} | 0.9 ± 0.01^{c} | 11.87 ± 0.73^{a} |
| C | 88.89 ± 1.92^{a} | 1.05 ± 0.05^{b} | 80.09 ± 4.65^{b} | 4.45 ± 0.41^{a} | 1.01 ± 0.06^{bc} | 11.73 ± 0.89^{a} |
| D | 84.44 ± 1.93^{b} | 0.99 ± 0.04^{b} | 74.46 ± 4.32^{c} | 4.02 ± 0.5^{a} | 1.35 ± 0.09^{a} | 11.42 ± 0.75^{ab} |

As shown, adding an appropriate concentration of salidroside fermentation broth to the feed can effectively reduce the mortality rate of grouper and increase its disease resistance. Experimental groups B and C showed significant effects, effectively increasing the weight of grouper and improving fat fullness.

Four groups of grouper organ homogenates and blood were collected after experimental feeding, and serum biochemistry, liver function, and immune indicators were tested. The results are shown in Table 10. It is noted that different lowercase letters on the shoulder of peer data indicate significant differences (P<0.05), while the same lowercase letters or no letters indicate no significant differences (P>0.05).

TABLE 10
The effects of salidroside on blood glucose, liver function, immunity, and oxidative damage in grouper

| Testing Indicators | A(control) | B | C | D |
|---|---|---|---|---|
| Alkaline phosphatase (ALP) (U/g) | 11.92 ± 2.68^{a} | 8.56 ± 0.59^{b} | 9.50 ± 0.63^{ab} | 9.00 ± 0.80^{b} |
| Acid Phosphatase (ACP) (U/g) | 259.64 ± 29.49^{a} | 210.05 ± 8.30^{b} | 249.14 ± 13.57^{a} | 241.74 ± 13.96^{ab} |
| Lysozyme (LZM) (U/g) | 7.70 ± 2.27^{c} | 10.63 ± 2.02^{a} | 8.84 ± 0.81^{b} | 8.47 ± 1.52^{b} |
| Superoxide Dismutase (SOD) (U/mg) | 18.26 ± 2.07^{c} | 26.47 ± 2.06^{a} | 21.42 ± 6.45^{b} | 18.67 ± 3.66^{c} |
| Catalase (CAT) (U/mg) | 12.95 ± 2.76^{b} | 18.62 ± 0.75^{a} | 12.44 ± 4.35^{b} | 9.67 ± 1.91^{b} |
| Lipase (LPS) (U/mg) | 4.95 ± 0.53^{b} | 2.86 ± 0.51^{c} | 3.42 ± 0.91^{bc} | 7.53 ± 2.19^{a} |
| Alanine Aminotransferase (ALT) (U/g) | 34.14 ± 4.44^{a} | 29.46 ± 1.26^{b} | 29.98 ± 3.26^{b} | 33.12 ± 4.2^{a} |
| Aspartate Transaminase (AST) (U/g) | 27.62 ± 1.19^{a} | 21.39 ± 0.96^{b} | 19.81 ± 1.13^{b} | 23.08 ± 3.91^{b} |
| Serum Glucose (mmol/mL) | 9.2 ± 1.31^{a} | 7.07 ± 2.52^{ab} | 4.53 ± 0.93^{bc} | 2.79 ± 0.66^{c} |

From the data, it can be seen that salidroside can significantly reduce the blood glucose levels of grouper, and the hypoglycemic effect is better with increasing dosage. This indicates that salidroside enhances the utilization of sugar by grouper, promotes blood glucose conversion, and thus can increase the amount of carbohydrates in feed to reduce feed consumption. Rhodiola glycoside significantly reduces alanine aminotransferase and aspartate aminotransferase, and adding Rhodiola glycoside to feed has a cytidine hepatoprotective effect. For indicators related to oxidative damage, both group B and group C have a good effect on reducing oxidative damage, especially group B has a better effect. Adding salidroside can reduce the levels of alkaline phosphatase and acid phosphatase, and increase the content of lysozyme, indicating that it can enhance the immunity of grouper.

### Example 8: Rhodiola Glycoside Fermentation Broth for South American White Shrimp Farming

Feeding plan: Control group A was only fed with basic feed, while groups B, C, and D were added with 10, 20, and 40 mg salidroside equivalent/kg feed, respectively. The experimental period was 8 weeks.

Breeding cycle: Two months, various growth indicators of South American white shrimp were tested, and the results are summarized in Table 11. It is noted that different lowercase letters on the shoulder of peer data indicate significant differences (P<0.05), while the same lowercase letters or no letters indicate no significant differences (P>0.05).

TABLE 11
Effect of Rhodiola rosea glycoside on growth performance
indicators of South American white shrimp

| | Group | | | |
|---|---|---|---|---|
| Test Indicators | A | B | C | D |
| Weight Gain Rate (%) | 42.84 ± 2.09^{b} | 45.87 ± 3.75^{b} | 53.04 ± 4.01^{a} | 44.53 ± 1.83^{b} |
| Specific Growth Rate (%/d) | 1.11 ± 0.05^{b} | 1.18 ± 0.08^{b} | 1.33 ± 0.08^{a} | 1.15 ± 0.04^{b} |
| Condition Factor (%) | 1.20 ± 0.04^{ab} | 1.22 ± 0.05^{ab} | 1.17 ± 0.05^{b} | 1.26 ± 0.02^{a} |
| Hepatosomatic Index (%) | 3.97 ± 0.27 | 3.93 ± 0.42 | 3.96 ± 0.25 | 4.06 ± 0.38 |

As shown in Table 11, adding an appropriate concentration of salidroside fermentation broth to the feed has a promoting effect on the growth of South American white shrimp. Compared with group A, the weight gain rate and specific growth rate of groups B and C were significantly improved, with group C showing the most significant improvement.

Four groups of South American white shrimp liver, pancreas, and blood were collected to detect serum biochemical indicators, liver function, and immune indicators. The results are shown in Table 12. It is noted that different lowercase letters on the shoulder of peer data indicate significant differences (P<0.05), while the same lowercase letters or no letters indicate no significant differences (P>0.05).

TABLE 12
Effects of salidroside on blood glucose, liver function, immunity,
and oxidative damage in South American white shrimp

| | Group | | | |
|---|---|---|---|---|
| Test Indicators | A | B | C | D |
| Glucose (GLU) (mmol/L) | 5.56 ± 1.51^{a} | 0.96 ± 0.21^{b} | 0.77 ± 0.06^{b} | 0.75 ± 0.10^{b} |
| Total Cholesterol (TCHO) (mmol/L)) | 0.47 ± 0.01^{a} | 0.41 ± 0.02^{b} | 0.47 ± 0.02^{a} | 0.29 ± 0.03^{c} |
| Low-Density Lipoprotein | 0.18 ± 0.02^{a} | 0.13 ± 0.02^{b} | 0.08 ± 0.01^{c} | 0.07 ± 0.01^{c} |
| Cholesterol (LDLC) (mmol/L) | | | | |
| High-Density Lipoprotein | 0.21 ± 0.01^{a} | 0.13 ± 0.01^{b} | 0.19 ± 0.02^{a} | 0.10 ± 0.01^{c} |
| Cholesterol (HDLC) (mmol/L) | | | | |
| Aspartate Aminotransferase (AST) (U/L) | 60.03 ± 2.8^{a} | 26.98 ± 0.04^{bc} | 30.87 ± 3.05^{b} | 23.68 ± 2.62^{c} |
| Alanine Aminotransferase (ALT) (U/L) | 109.68 ± 5.4^{a} | 91.62 ± 3.27^{b} | 87.91 ± 2.33^{b} | 67.90 ± 1.52^{c} |
| Superoxide Dismutase (SOD) (U/mg) | 4.87 ± 0.31^{d} | 7.02 ± 0.07^{c} | 10.15 ± 0.28^{a} | 7.86 ± 0.72^{b} |
| Catalase (CAT) (U/mg) | 17.07 ± 2.55^{a} | 24.23 ± 3.53^{b} | 33.37 ± 0.33^{c} | 24.86 ± 0.78^{b} |
| Malondialdehyde (MDA) (nmol/mg) | 20.24 ± 1.30^{a} | 8.08 ± 1.06^{b} | 5.54 ± 0.37^{c} | 9.82 ± 2.97^{b} |
| Total Antioxidant Capacity (T-AOC) (mmol/g) | 25.96 ± 0.34^{bc} | 23.73 ± 1.86^{c} | 46.66 ± 0.9^{a} | 27.94 ± 0.94^{b} |
| Acid Phosphatase (ACP) (U/g) | 352.04 ± 49.57^{b} | 604.60 ± 11.23^{a} | 564.60 ± 31.96^{a} | 366.72 ± 28.31^{b} |
| Alkaline Phosphatase (AKP) (U/g) | 386.87 ± 16.54^{c} | 587.38 ± 17.74^{a} | 552.81 ± 25.54^{ab} | 486.16 ± 8.88^{b} |

As shown in Table 12, salidroside can significantly reduce the blood glucose content, total cholesterol, high-density lipoprotein cholesterol, and low-density lipoprotein cholesterol content of South American white shrimp, and the effect becomes more pronounced with increasing dosage. Rhodiola rosea glycoside can reduce the activity of aspartate aminotransferase and alanine aminotransferase in the blood of South American white shrimp, protecting liver health. At the same time, it can significantly increase the activity of superoxide dismutase and catalase in the liver and pancreas of South American white shrimp, enhancing the body's antioxidant capacity. Rhodiola rosea glycoside can significantly increase the activity of acid phosphatase and alkaline phosphatase in the liver and pancreas of South American white shrimp, enhancing the body's immunity.

Four groups of Litopenaeus vannamei were subjected to low-oxygen and desiccation tolerance tests. The test results are shown in Table 13. It is noted that different lowercase letters on the shoulder of peer data indicate significant differences (P<0.05), while the same lowercase letters or no letters indicate no significant differences (P>0.05).

TABLE 13
The effects of salidroside on the low-oxygen and
desiccation tolerance of Litopenaeus vannamei

Group

| Items | A | B | C | D |
|---|---|---|---|---|
| Desiccation (Time | 15.07 ± | 20.00 ± | 20.67 ± | 15.67 ± |
| to Death, min) | 1.03^{b} | 0.72^{a} | 1.14^{a} | 1.10^{b} |
| Hypoxia (Time | 25.73 ± | 31.73 ± | 38.40 ± | 30.67 ± |
| to Death, min) | 1.22^{c} | 3.35^{b} | 4.21^{a} | 0.83^{bc} |

As can be seen from Table 13, salidroside can significantly improve the low-oxygen tolerance and desiccation resistance of Litopenaeus vannamei, which is of positive significance for situations with insufficient dissolved oxygen and long-distance transportation.

In the foregoing description, numerous specific details are set forth, such as specific materials, dimensions, processes parameters, etc., to provide a thorough understanding of the embodiments of the present disclosure. The particular features, structures, materials, or characteristics may be combined in any suitable manner in one or more embodiments. The words “example” or “exemplary” are used herein to mean serving as an example, instance, or illustration. Any aspect or design described herein as an “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the use of the terms “a,” “an,” “the,” and similar referents in the context of describing the materials and methods discussed herein (especially in the context of the following claims) are to be construed to cover both the singular and the plural, unless otherwise indicated herein or clearly contradicted by context.

Recitations of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Reference throughout this specification to “one embodiment,” “certain embodiments,” “one or more embodiments,” “an embodiment,” or “some embodiments” means that a particular feature, structure, material, or characteristic described in connection with the embodiment is included in at least one embodiment of the present disclosure. Thus, the appearances of the phrases such as “in one or more embodiments,” “in certain embodiments,” “in one embodiment,” or “in an embodiment” in various places throughout this specification are not necessarily referring to the same embodiment of the present disclosure. Furthermore, the particular features, structures, materials, or characteristics may be combined in any suitable manner in one or more embodiments.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Although the embodiments disclosed herein have been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents, and the above-described embodiments are presented for the purposes of illustration and not of limitation.

What is claimed is:

1. A feed additive comprising a fermentation broth of a microbial strain.

2. The feed additive of claim 1, wherein the microbial strain is a yeast strain.

3. The feed additive of claim 2, wherein the yeast strain is a Saccharomyces cerevisiae or a modified version thereof.

4. The feed additive of claim 1, wherein the feed additive maintains at least 95% chemical stability after about six months storage at room temperature or approximately 20-25° C.

5. The feed additive of claim 1, wherein the fermentation broth comprises salidroside.

6. The feed additive of claim 2, wherein the fermentation broth comprises whole yeast cells, water, media, and biomolecules excreted by the yeast strain.

7. The feed additive of claim 1, wherein the fermentation broth is produced by a process comprising activating a yeast strain, performing seed cultivation, and performing fermentation under controlled conditions of temperature, aeration, pressure, stirring speed, dissolved oxygen, and pH.

8. The feed additive of claim 1, wherein the fermentation broth is subjected to high-temperature inactivation conditions, comprising maintaining the fermentation broth at a temperature of about 80° C. to about 120° C. for about 0.5 hour to about 2 hours.

9. An animal feed formulation comprising:
the feed additive of claim 1; and
at least one carrier excipient.

10. An animal feed formulation comprising salidroside present from about 10 mg/kg to about 100 mg/kg, calculated based on total weight of the animal feed formulation and at least one carrier excipient.

11. The animal feed formulation of claim 10, further comprising at least one additive for animal husbandry selected from astaxanthin, oligochitosan, fructooligosaccharides, oligosaccharides, galactooligosaccharides, conjugated linoleic acid, alginate oligosaccharides, curcumin, bile acids, citric acid ketone, plant charcoal black, thirty-seven peptides of dry grass, berberine, red yeast, lutein, β-carotene, chili red, neomethyl hesperidin dihydrochalcone, allicin, monosodium glutamate, disodium 5′-inosinate, disodium 5′-guanylate, tea polyphenols, and combinations thereof.

12. The animal feed formulation of claim 10, further comprising an acidity regulator selected from formic acid, ammonium formate, calcium formate, acetic acid, sodium diacetate, propionic acid, ammonium propionate, sodium propionate, calcium propionate, butyric acid, sodium butyrate, lactic acid, benzoic acid, sodium benzoate, sorbic acid, sodium sorbate, potassium sorbate, fumaric acid, citric acid, potassium citrate, sodium citrate, calcium citrate, tartaric acid, malic acid, phosphoric acid, sodium hydroxide, sodium bicarbonate, potassium chloride, sodium carbonate, calcium acetate, sodium pyrophosphate, sodium tripolyphosphate, sodium hexametaphosphate, sodium metabisulfite, monosodium dihydrogen pyrophosphate, potassium diformate, ammonium chloride, sodium sulfite, and combinations thereof.

13. A method of forming a fermentation broth of a microbial strain comprising:
activating the microbial strain, performing seed culture, and preparing a fermentation culture.

14. The method of claim 13, further comprising:
subjecting the fermentation broth to high-temperature inactivation conditions, wherein the high-temperature inactivation conditions comprise maintaining the fermentation broth at about 80° C. to about 110° C. for about 1 hour to about 2 hours.

15. The method of claim 13, wherein the pH of the salidroside fermentation broth is adjusted to a range of about 2.5 to about 3.5.

16. The method of claim 13, wherein the microbial strain is a yeast strain, and wherein the yeast strain is a Saccharomyces cerevisiae or a modified version thereof.

17. The method of claim 13, wherein the fermentation broth comprises salidroside.

18. A method of feeding an animal comprising:
administering to the animal a feed additive or feed formulation of claim 1.

19. The method of claim 18, wherein:
the animal is livestock selected from cow, bison, buffalo, hogs, pigs, boar, sheep, goats, rabbits, mink, deer, alligator, snakes, horses, donkeys, chickens, ostrich, turkeys, ducks, geese, quails, game hens, and broiler chicken; or
the animal is a crustacean selected from lobsters, crabs, shrimp, prawns and crayfish; or
the animal is a fish selected from salmon, trout, whitefish, grouper, sea bass, catfish, tuna, carp, and cod.

20. The method of claim 19, wherein the administering is performed through liquid administration or by combination with an animal feed formulation.
